(19) United States
(12) Patent Application Publication
(10) Pub. No.: US 2024/0342006 A1
(43) Pub. Date: Oct. 17, 2024

(54) METHOD FOR PROVIDING CONTROL DATA FOR AN OPHTHALMOLOGICAL LASER OF A TREATMENT APPARATUS FOR AVOIDING AN OPAQUE BUBBLE LAYER

(71) Applicant: SCHWIND EYE-TECH-SOLUTIONS GMBH, Kleinostheim (DE)

(72) Inventors: Samuel ARBA MOSQUERA, Aschaffenburg (DE); Mario SHRAIKI, Ober-Ramstadt (DE); Nico TRIEFENBACH, Mainaschaff (DE)

(21) Appl. No.: 18/631,297

(22) Filed: Apr. 10, 2024

(30) Foreign Application Priority Data
Apr. 11, 2023 (DE) 10 2023 109 045.3

(51) Int. Cl. A61F 9/008; A61B 17/00; A61B 18/00

(52) U.S. Cl. CPC A61F 9/00804; A61F 9/00827; A61B 2017/0019; A61B 2018/00702; A61F 2009/00872; A61F 2009/00878

(57) ABSTRACT

A method for providing control data for an ophthalmological laser of a treatment apparatus for avoiding an opaque bubble layer in a cornea is provided. There is effected determining a treatment area in the cornea by predetermined examination data, wherein first irradiation parameters for providing an optical breakdown are set; determining an initial irradiation area in or adjoining to the treatment area, in which it is begun with an irradiation of the cornea, wherein second irradiation parameters for the optical breakdown are set for the initial irradiation area, by which a reduced power density is generated; wherein a positioning of at least a first laser pulse path is set adjoining to the initial irradiation area for a subsequent irradiation of the treatment area. Finally, providing the control data for the ophthalmological laser, which includes the treatment area and the initial irradiation area with the respective irradiation parameters, is affected.

### FIELD

The invention relates to a method for providing control data for an ophthalmological laser of a treatment apparatus for avoiding an opaque bubble layer in a cornea. Furthermore, the invention relates to a control device, which is configured to perform the method, to a treatment apparatus with such a control device, to a computer program comprising commands, which cause the treatment apparatus to execute the method, and to a computer-readable medium, on which the computer program is stored.

### BACKGROUND

Treatment apparatuses and methods for controlling ophthalmological lasers for correcting an optical visual disorder and/or pathologically or unnaturally altered areas of the cornea are known in the prior art. Therein, pulsed lasers and a beam focusing device can for example be formed such that laser pulses effect a photodisruption and/or ablation in a focus situated within the organic tissue, to remove a tissue, in particular a tissue lenticule, from the cornea.

Upon irradiation of the cornea, in particular upon an incision surface generation in the cornea, however, it can occur that an opaque bubble layer is generated in the cornea by the laser pulses, which can influence an optical breakdown threshold (“laser induced optical breakdown”) for further pulses. This opaque bubble layer often arises at the beginning of the treatment. In particular, the development of the opaque bubble layer can be ascribed to the fact that an excessive portion of energy changes an adjoining tissue structure in the cornea and thus generates blurs. This has the disadvantage that a sight is restricted at these locations besides the impact on further pulses.

### SUMMARY

Therefore, it is the object of the invention to provide control data for treating a cornea, by which a generation of an opaque bubble layer can be minimized or avoided.

This object is solved by the independent claims. Advantageous embodiments of the invention are disclosed in the examples provided herein, the following description as well as the figures.

The invention is based on the idea that the tissue is irradiated with a reduced energy in an initial irradiation area, which is also above the optical breakdown threshold, but has a low energy excess, such that the generation of the opaque bubble layer is reduced. Thus, holes can be generated in the initial irradiation area of the cornea, which can have an insufficient quality for an incision surface generation, but a space can be created by these holes, in which the excessive energy of subsequent pulses, which is in particular present in the form of a pressure wave and/or heat, can be dissipated. Thus, it can be avoided that the intact corneal tissue does not have to absorb this energy, which is the cause of the generation of the opaque bubble layer. Irradiation positions adjoining to this initial irradiation area can then be treated with an increased or an originally planned energy, which is optimized for the separation of the corneal tissue, wherein an energy of the generated optical breakdown in these adjoining irradiation positions can propagate towards the initial irradiation area, and the generation of an opaque bubble layer can thus be reduced. In particular, further pulses are then planned adjoining to the already irradiated irradiation positions, whereby sufficient space for dissipating the excessive energy is always present.

An aspect of the invention relates to a method for providing control data for an ophthalmological laser of a treatment apparatus for avoiding an opaque bubble layer in a cornea, wherein the method can be performed by a control device. Therein, an appliance or an appliance component, in particular a processor or microprocessor, is to be understood by a control device, by which the following steps can be executed and/or planned.

There is effected determining a treatment area in the cornea by predetermined examination data, wherein first irradiation parameters for providing an optical breakdown are set for the treatment area, determining an initial irradiation area in or adjoining to the treatment area, in which an irradiation of the cornea is started, wherein second irradiation parameters for the optical breakdown are set for the initial irradiation area, wherein a power density reduced compared to the first irradiation parameters is generated in the cornea by the second irradiation parameters, wherein a positioning of at least a first laser pulse path adjoining to the initial irradiation area is set for an irradiation of the treatment area by the first irradiation parameters subsequent to the initial irradiation area. Finally, providing the control data for the ophthalmological laser, which includes the treatment area and the initial irradiation area with the respective irradiation parameters, is affected.

In other words, the treatment area in the cornea can first be set, which is to be irradiated for the correction of a visual disorder. An ablation profile and/or incision surfaces, in particular anterior and posterior incision surfaces for generating a volume body, by which an optical zone with a transition zone adjoining thereto is provided, can for example be determined as the irradiation area. In particular, this treatment area can be planned due to predetermined examination data, such as for example a topography and/or an aberration measurement. For irradiation of this treatment area, first irradiation parameters can be set, such that a power density of the first irradiation parameters is above a threshold of an optical breakdown, wherein the threshold of the optical breakdown can be predetermined or preset. The first irradiation parameters are preferably optimized for the separation of the corneal tissue.

In the treatment area, an initial irradiation area can be defined, which is in the treatment area or adjoins thereto. In this initial irradiation area, the positioning of the first laser pulses can be planned at the beginning of the treatment, for example an initial laser pulse path or an isolated area. The irradiation of the initial irradiation area can be planned with second irradiation parameters, wherein the second irradiation parameters generate a power density reduced compared to the first irradiation parameters, but are further above the threshold for the optical breakdown. The respective irradiation parameters can for example include a laser pulse energy, a repetition rate of the laser and/or a spatial distance of adjacent laser pulses to generate the respective power density. Thus, the initial irradiation area can include a subset of the treatment area, for example a circular segment, on which a first laser pulse path is generated. For example, the treatment area can be a lenticule with an optical zone and transition zone, wherein the initial irradiation area can be a laser pulse path adjoining to the lenticule, that is outside of the transition zone. In particular, the initial irradiation area can extend the lenticule by not more than one millimeter. For example, the initial irradiation area can be within the planned treatment area.

The positioning of subsequent laser pulses, in particular a first laser pulse path, after the initial irradiation area, which are performed in the treatment area with the first irradiation parameters, can then be planned adjoining to the initial irradiation area, to thus create a dissipation of excessive energy into the space, which is provided by the initial irradiation area. This means, after the initial irradiation area has been irradiated with the second irradiation parameters, an irradiation of the treatment area subsequent thereto can be begun, wherein a first laser pulse path is thereto set adjoining to the initial irradiation area. In particular, a second laser pulse path can then adjoin to the first laser pulse path, wherein this scheme can then be continued until the entire treatment area, for example the lenticule, is generated.

Finally, control data can be provided for the ophthalmological laser, which includes the treatment area and the initial irradiation area with the respective irradiation parameters. Then, the laser can be controlled with the control data in particular for correcting a visual disorder and for avoiding an opaque bubble layer.

By the invention, the advantage arises that holes can be generated in the cornea by the initial irradiation area, into which excessive energy, which is above an optical breakdown threshold and which can in particular be present at the first irradiation parameters, can be dissipated. Thus, it can be avoided that the intact corneal tissue absorbs too much energy, which reduces or avoids the generation of the opaque bubble layer in the areas, which adjoin to the initial irradiation area. In that the subsequent laser pulse paths may be provided adjoining to the initial irradiation area or adjoining to a respectively generated laser pulse path, the generation of the opaque bubble layer can be avoided for the entire treatment area. Accordingly, a better treatment result can be achieved.

The invention also includes embodiments, by which additional advantages arise.

In an embodiment a power density reduced by at least 20%, in particular a power density reduced by 50%, compared to the first irradiation parameters is generated by the second irradiation parameters. In other words, the second irradiation parameters can generate a power density of 80%, in particular 50%, of the first irradiation parameters. However, the power density may be further above the optical breakdown threshold. Thus, it can be achieved that holes are generated in the cornea in the initial irradiation area, but excessive energy, which generates an opaque bubble layer, is avoided. Herein, the energy per area unit or volume unit, which is generated by the laser pulses in the cornea, is meant by the power density. Hereby, the advantage arises that suitable irradiation parameters for generating the initial irradiation area can be provided.

In another embodiment a pulse energy is reduced for the second irradiation parameters. This means that the pulse energy of the second irradiation parameters can be reduced compared to the pulse energy of the first irradiation parameters to reduce the power density, with which the initial irradiation area is irradiated. Thus, the advantage arises that a reduced power density can be provided, which obviates the generation of an opaque bubble layer.

In another embodiment a laser pulse distance is increased for the second irradiation parameters. This means that a laser pulse distance of the second irradiation parameters is increased compared to a laser pulse distance of the first irradiation parameters. A spatial laser pulse distance, thus the distance of the laser pulse positions in the cornea, is meant by laser pulse distance. By increasing the spatial laser pulse distance, the power density, thus the energy per area, is reduced and the development of the opaque bubble layer is thus avoided.

In another embodiment a distance of adjacent laser pulses on a laser pulse path and/or a distance of adjacent laser pulse paths is increased for increasing the laser pulse distance. This means that a laser pulse path can be provided in the initial irradiation area, wherein a distance of adjacent laser pulses on this laser pulse path is increased. Alternatively or additionally, multiple laser pulse paths can be provided in the initial irradiation area, which are arranged next to each other, wherein a distance of these adjacent laser pulse paths can be increased to reduce the power density. Hereby, suitable configurations for providing the second irradiation parameters can be achieved.

In another embodiment a repetition rate of the laser is reduced for the second irradiation parameters. In other words, a repetition frequency of the laser of the second irradiation parameters can be reduced compared to a repetition rate of the laser of the first irradiation parameters, for example by a pulse picker, to reduce the power density for irradiating the initial irradiation area. Hereby, a further suitable form of configuration of the second irradiation parameters can be achieved.

In another embodiment the initial irradiation area is again irradiated by the first irradiation parameters after at least the first laser pulse path has been generated in the treatment area. In other words, it can be provided that the initial irradiation area is first irradiated by the second irradiation parameters and subsequently the first laser pulse path adjoining to this initial irradiation area is irradiated by the first irradiation parameters. If this first laser pulse path is generated by the first irradiation parameters, the initial irradiation area can be again irradiated, wherein it is then irradiated by the first irradiation parameters, which have a higher power density than the second irradiation parameters. This is particularly advantageous if the initial irradiation area is within the treatment area. Thus, holes can first be generated in the initial irradiation area, into which the energy of the first laser pulse path can be dissipated, wherein this initial irradiation area can subsequently again be incised with the optimized first irradiation parameters, since the excessive energy can then be dissipated into the area, which is provided by the first laser pulse path. Hereby, the advantage arises that the entire treatment area, in particular including the initial irradiation area, can be irradiated with optimized irradiation parameters and thus the corneal tissue can be separated.

In another embodiment a lenticule is defined in the cornea as the treatment area, wherein the initial irradiation area is planned adjoining to the lenticule viewed in the radial direction. In particular, the initial irradiation area can be planned surrounding the lenticule viewed in radial direction, wherein the subsequent laser pulse paths for irradiating the treatment area are then generated adjoining to it, in particular in the form of concentric circular paths, the radius of which is reduced with each new path, and/or in the form of a spiral path from the outside up to the center. Accordingly, an irradiation is effected from an outer side of the lenticule to the inside. Hereby, the advantage arises that a preferred irradiation scheme can be provided.

In another embodiment a lenticule with an anterior and posterior interface is defined as the irradiation area, wherein the initial irradiation area is planned as an annulus around a center of the respective interface. This means that the initial irradiation area is provided in the treatment area, which extends annularly around a center of the respective interface. Thus, the annular initial irradiation area can for example first be irradiated by the second irradiation parameters and thereafter laser pulse paths starting therefrom by the first irradiation parameters to the center of the respective interface. In the center, the direction can change to the outside, whereby the annular initial irradiation area is also again irradiated by the first irradiation parameters. By this embodiment, a further advantageous configuration of an irradiation scheme for avoiding the opaque bubble layer arises.

A further aspect of the invention relates to a method for controlling a treatment apparatus. Therein, the method includes the method steps of at least one embodiment of a method as it was previously described. Furthermore, the method for controlling the treatment apparatus also includes the step of transferring the provided control data to at least one ophthalmological laser of the treatment apparatus and controlling the treatment apparatus and/or the laser with the control data.

The respective method can include at least one additional step, which is executed if and only if an application case or an application situation occurs, which has not been explicitly described here. For example, the step can include the output of an error message and/or the output of a request for inputting a user feedback. Additionally or alternatively, it can be provided that a default setting and/or a predetermined initial state are adjusted.

A further aspect of the invention relates to a control device, which is formed to perform the steps of at least one embodiment of one or both of the previously described methods. Thereto, the control device can comprise a computing unit for electronic data processing such as for example a processor. The computing unit can include at least one microcontroller and/or at least one microprocessor. The computing unit can be configured as an integrated circuit and/or microchip. Furthermore, the control device can include an (electronic) data memory or a storage unit. A program code can be stored on the data memory, by which the steps of the respective embodiment of the respective method are encoded. The program code can include the control data for the respective laser. The program code can be executed by the computing unit, whereby the control device is caused to execute the respective embodiment. The control device can be formed as a control chip or control unit. The control device can for example be encompassed by a computer or computer cluster.

A further aspect of the invention relates to a treatment apparatus with at least one eye surgical or ophthalmological laser and a control device, which is formed to perform the steps of at least one embodiment of one or both of the previously described methods. The respective laser can be formed to at least partially separate a predefined corneal volume with predefined interfaces of a human or animal eye by optical breakdown, in particular at least partially separate it by photodisruption and/or to ablate corneal layers by (photo)ablation and/or to effect a laser-induced refractive index change in the cornea and/or the eye lens and/or to increase a crosslinking of the cornea.

In a further advantageous embodiment of the treatment apparatus according to the invention, the laser can be suitable to emit laser pulses in a wavelength range between 300 nm and 1400 nm, for example between 900 nm and 1200 nm, at a respective pulse duration between 1 fs and 1 ns, for example between 10 fs and 10 ps, and a repetition frequency of greater than 10 kilohertz (kHz), for example between 100 kHz and 100 megahertz (MHz). The use of such lasers in the method according to the invention additionally has the advantage that the irradiation of the cornea does not have to be effected in a wavelength range below 300 nm. This range is subsumed by the term “deep ultraviolet” in the laser technology. Thereby, it is advantageously avoided that an unintended damage to the cornea is effected by these very short-wavelength and high-energy beams. Photodisruptive and/or ablative lasers of the type used here usually input pulsed laser radiation with a pulse duration between 1 fs and 1 ns into the corneal tissue. Thereby, the power density of the respective laser pulse required for the optical breakdown can be spatially narrowly limited such that a high incision accuracy is allowed in the generation of the interfaces. In particular, the range between 700 nm and 780 nm can also be selected as the wavelength range.

In a further advantageous embodiment of the treatment apparatus according to the invention, the control device can comprise at least one storage device for at least temporary storage of at least one control dataset, wherein the control dataset or datasets include(s) control data for positioning and/or for focusing individual laser pulses in the cornea; and can comprise at least one beam device for beam guidance and/or beam shaping and/or beam deflection and/or beam focusing of a laser beam of the laser.

A further aspect of the invention relates to a computer program. The computer program includes commands, which for example form a program code. The program code can include at least one control dataset with the respective control data for the respective laser. Upon execution of the program code by a computer or a computer cluster, it is caused to execute the previously described method or at least one embodiment thereof.

A further aspect of the invention relates to a computer-readable medium (storage medium), on which the above mentioned computer program and the commands thereof, respectively, are stored. For executing the computer program, a computer or a computer cluster can access the computer-readable medium and read out the content thereof. The storage medium is for example formed as a data memory, in particular at least partially as a volatile or a non-volatile data memory. A non-volatile data memory can be a flash memory and/or an SSD (solid state drive) and/or a hard disk. A volatile data memory can be a RAM (random access memory). For example, the commands can be present as a source code of a programming language and/or as assembler and/or as a binary code.

Further features and advantages of one of the described aspects of the invention can result from the embodiments of another one of the aspects of the invention. Thus, the features of the embodiments of the invention can be present in any combination with each other if they have not been explicitly described as mutually exclusive.

### BRIEF DESCRIPTION OF THE DRAWINGS

In the following, additional features and advantages of the invention are described in the form of advantageous execution examples based on the figure(s). The features or feature combinations of the execution examples described in the following can be present in any combination with each other and/or the features of the embodiments. This means, the features of the execution examples can supplement and/or replace the features of the embodiments and vice versa. Thus, configurations are also to be regarded as encompassed and disclosed by the invention, which are not explicitly shown or explained in the figures, but arise from and can be generated by separated feature combinations from the execution examples and/or embodiments. Thus, configurations are also to be regarded as disclosed, which do not comprise all of the features of an originally formulated claim or extend beyond or deviate from the feature combinations set forth in the relations of the claims. To the execution examples, there shows:

FIG. 1 a schematic representation of a treatment apparatus according to an exemplary embodiment.

FIG. 2 a schematic method diagram for providing control data according to an exemplary embodiment.

FIG. 3 a schematic representation of a treatment area with an initial irradiation area according to an exemplary embodiment.

FIG. 4 a schematic representation of a treatment area with an initial irradiation area according to a further exemplary embodiment.

In the figures, identical or functionally identical elements are provided with the same reference characters.

### DETAILED DESCRIPTION

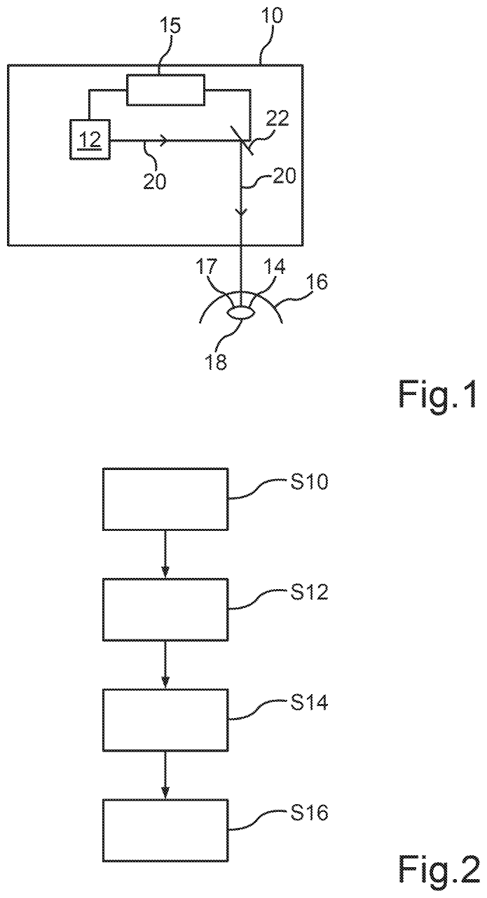

FIG. 1 shows a schematic representation of a treatment apparatus 10 with an ophthalmological laser 12 for removing a tissue 14 from a human or animal cornea 16 by photodisruption and/or ablation. For example, the tissue 14 can represent a lenticule 14 or also volume body, which can be separated from the cornea 16 by the eye surgical laser 12 for correcting a visual disorder. In particular, an anterior interface 17 and a posterior interface 18 can be defined as a treatment area, which together form the lenticule 14, wherein the laser pulses can be planned in the treatment area. The treatment area, for example the position of the interfaces 17, 18, can be defined by a control device 15 in the form of control data, wherein the laser 12 emits pulsed laser pulses in a pattern predefined by the control data into the cornea 16 of the eye by the control data, to generate the lenticule 14. In particular, irradiation parameters can also be provided in the control data, which are in particular optimized for separating the lenticule 14, wherein the irradiation parameters can include a laser pulse energy, a repetition rate of the laser and/or laser pulse distances. In this figure, the control device 15 is represented as part of the treatment apparatus 10, wherein the control device 15 can alternatively also be a control device 15 external with respect to the treatment apparatus 10, for example in the form of a planning device.

Furthermore, FIG. 1 shows that the laser beam 20 generated by the laser 12 can be deflected towards the cornea 16 by a beam deflection device 22 such as for example a rotation scanner, to generate the lenticule 14. The beam deflection device 22 can also be controlled by the control device 15.

In particular, the illustrated laser 12 can be a photodisruptive and/or photoablative laser, which is formed to emit laser pulses in a wavelength range between 300 nanometers and 1400 nanometers, for example between 700 nanometers and 1200 nanometers, at a respective pulse duration between 1 femtosecond and 1 nanosecond, for example between 10 femtoseconds and 10 picoseconds, and a repetition frequency of greater than 10 kilohertz, for example between 100 kilohertz and 100 megahertz. In addition, the control device 15 optionally comprises a storage device (not illustrated) for at least temporary storage of at least one control dataset, wherein the control dataset or datasets include(s) control data for positioning and/or for focusing individual laser pulses in the cornea.

In generating the lenticule 14, it can occur in particular at the beginning of the treatment that an opaque bubble layer is generated in the cornea 16. This effect can be due to the fact that excessive energy above a threshold for the optical breakdown is dissipated into the surrounding corneal tissue, which blurs due to that. By these blurs, the threshold of the optical breakdown for subsequent pulses can change in these areas, which is disadvantageous for the treatment. In order to reduce or avoid the development of this opaque bubble layer, the method shown in FIG. 2 can be performed.

In FIG. 2, a schematic method diagram for providing control data for the ophthalmological laser 12 of the treatment apparatus 10 for avoiding the opaque bubble layer in the cornea 16 according to an exemplary embodiment is illustrated.

In a step S10, a treatment area 17, 18 in the cornea 16 can be ascertained from predetermined examination data, wherein the treatment area can for example be provided by a laser pulse placement for generating the anterior interface 17 and/or the posterior interface 18 of the lenticule 14. For this treatment area 17, 18, first irradiation parameters can be set, by which an optical breakdown can be generated in the corneal tissue and which are preferably optimized for the separation of the corneal tissue. In particular, the irradiation parameters can include a laser pulse energy, a repetition rate of the laser 12 and/or a spatial laser pulse distance.

In a step S12, an initial irradiation area 24 can be ascertained, which is in the treatment area 17, 18 or adjoining to it. The initial irradiation area 24 can include at least one laser pulse path, in particular, multiple laser pulse paths, which are first generated in the cornea 16, wherein second irradiation parameters are used hereto, by which an optical breakdown can be generated, but which generate a reduced power density in the cornea 16 compared to the first irradiation parameters. Hereto, the pulse energy of the laser pulses may be reduced and/or a repetition rate of the laser can be reduced and/or a laser pulse distance, in particular a distance of adjacent laser pulses on a laser pulse path and/or a distance of adjacent laser pulse paths, can be increased to reduce the power density in the initial irradiation area 24 compared to the treatment area 17, 18. In particular, the power density of the second irradiation parameters can be reduced by at least 20%, for example by 50%, compared to the first irradiation parameters.

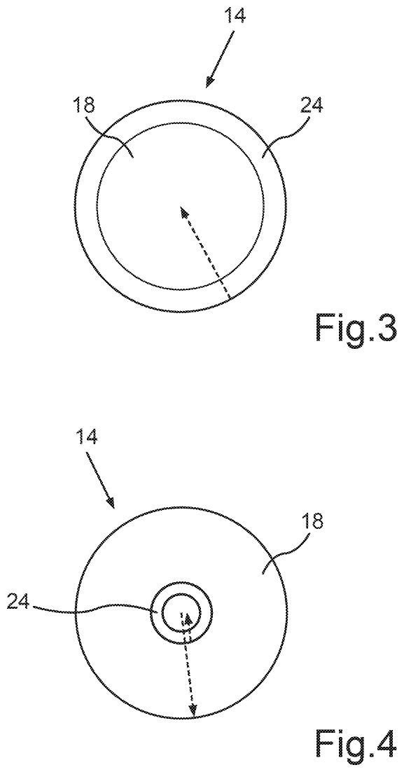

In FIG. 3, a schematic representation of a treatment area of the lenticule 14, in this example a top view to the posterior interface 18, and of an initial irradiation area 24 adjoining thereto, is shown according to an exemplary embodiment. The initial irradiation area 24 can be planned adjoining to the posterior interface 18 viewed in radial direction, wherein it can similarly adjoin to the anterior interface 17 not illustrated in this figure. In this example, the posterior interface 18 represents the treatment area, which is treated with the first irradiation parameters, wherein the initial irradiation area 24 is irradiated with the second irradiation parameters before the irradiation of the posterior interface 18. Herein, the second irradiation parameters can be configured to “perforate” the initial irradiation area 24 to provide spaces for dissipating the excessive energy.

With the arrangement of the initial irradiation area 24 at the edge of the lenticule 14, an irradiation of the interface 18 from the outside to the inside may be performed, which is indicated by the dashed arrow, such that the initial irradiation area 24 is first irradiated with the second irradiation parameters and subsequently an irradiation of the treatment area subsequent to the initial irradiation area 24 by the first irradiation parameters in a step S14, wherein a first laser pulse path adjoins to the initial irradiation area 24 in the treatment area. Accordingly, an irradiation from the outside to the inside is effected, wherein after generating the interface 18 or after generating the first laser pulse path, the initial irradiation area is for example again irradiated by the first irradiation parameters to separate the corneal tissue also in this area.

In FIG. 4, a further configuration of the initial irradiation area 24 is illustrated in the treatment area of the lenticule 14. Here too, an exemplary top view to the posterior interface 18 is shown, wherein a corresponding positioning of the initial irradiation area 24 can also be performed on the anterior interface 17. In this embodiment, the initial irradiation area 24 is provided as an annulus around a center of the posterior interface 18, which can be irradiated with the reduced second irradiation parameters at the beginning of the treatment. Subsequently, the first laser pulse path can be generated or planned in the treatment area towards the center of the interface 18 by the first irradiation parameters in the step S14, which is indicated by the dashed arrow towards the center. Therein, the laser pulse paths may be provided in the form of concentric or spiral paths. After reaching the center, the irradiation direction can change to the outside, whereby the annular initial irradiation area 24 can also be again irradiated by the first irradiation parameters and thus be separated.

Finally, control data, which includes the treatment area and the initial irradiation area 24 with the first and second irradiation parameters, can be provided for the ophthalmological laser 12 in a step S16.

Overall, the examples show, how a reduction or avoidance of an opaque bubble layer can be achieved in the treatment of a cornea 16 with an ophthalmological laser 12.

1. A method for providing control data for an ophthalmological laser of a treatment apparatus for avoiding an opaque bubble layer in a cornea, wherein the method comprises the following steps performed by a control device:
determining a treatment area in the cornea by predetermined examination data, wherein first irradiation parameters for providing an optical breakdown are set for the treatment area;
determining an initial irradiation area in or adjoining to the treatment area, in which an irradiation of the cornea is started, wherein second irradiation parameters for the optical breakdown are set for the initial irradiation area, wherein a power density reduced compared to the first irradiation parameters is generated by the second irradiation parameters;
wherein a positioning of at least a first laser pulse path adjoining to the initial irradiation area is set for an irradiation of the treatment area by the first irradiation parameters subsequent to the initial irradiation area;
providing the control data for the ophthalmological laser, which includes the treatment area and the initial irradiation area with the respective irradiation parameters.

2. The method according to claim 1, wherein a power density reduced by at least 20%, in particular a power density reduced by 50%, compared to the first irradiation parameters is generated by the second irradiation parameters.

3. The method according to claim 1, wherein a pulse energy is reduced for the second irradiation parameters.

4. The method according to claim 1, wherein a laser pulse distance is increased for the second irradiation parameters.

5. The method according to claim 4, wherein the laser pulse distance includes a distance of adjacent laser pulses on a laser pulse path and/or a distance of adjacent laser pulse paths.

6. The method according to claim 1, wherein a repetition rate of the laser is reduced for the second irradiation parameters.

7. The method according to claim 1, wherein the initial irradiation area is again irradiated by the first irradiation parameters after at least the first laser pulse path has been generated in the treatment area.

8. The method according to claim 1, wherein a lenticule is defined in the cornea as the treatment area, wherein the initial irradiation area is planned adjoining to the lenticule viewed in radial direction.

9. The method according to claim 1, wherein a lenticule with an anterior and posterior interface is defined as the irradiation area, wherein the initial irradiation area is planned as an annulus around a center of the respective interface.

10. A method for controlling a treatment apparatus, wherein the method comprises the following steps:
the method steps of the method according to claim 1, and
transferring the provided control data to a respective ophthalmological laser of the treatment apparatus.

11. A control device, which is configured to perform a respective method according to claim 1.

12. A treatment apparatus with at least one ophthalmological laser for the separation of a corneal volume with predefined interfaces of a human or animal eye by optical breakdown, in particular by photodisruption and/or photoablation, and at least one control device according to claim 11.

13. A non-transitory computer-readable medium configured for storing a computer program, the computer program comprising commands which cause a treatment apparatus to execute the method according to claim 1.

14. (canceled)
